(19) United States
(12) Patent Application Publication
(10) Pub. No.: US 2024/0382266 A1
(43) Pub. Date: Nov. 21, 2024

(54) MEDICAL INSTRUMENT, MEDICAL ASSISTANCE SYSTEM, METHOD FOR DETERMINING POSITION AND COMPUTER-READABLE STORAGE MEDIUM

(71) Applicant: B. Braun New Ventures GmbH, Freiburg im Breisgau (DE)

(72) Inventor: Jean Stawiaski, Kirchzarten (DE)

(21) Appl. No.: 18/659,549

(22) Filed: May 9, 2024

(30) Foreign Application Priority Data
May 15, 2023 (DE) 10 2023 112 754.3

(51) Int. Cl. A61B 34/20; A61B 90/00

(52) U.S. Cl. CPC A61B 34/20; A61B 90/36; A61B 2034/2051; A61B 2034/2055

(57) ABSTRACT

A medical instrument for a surgical assistance system includes a handle; an end section with a working point, in particular a tip; and a first reference system arranged in a predetermined first position relative to the working point, in particular rigidly attached to the handle, and which is adapted for determining a condition and/or an orientation of the end section with the working point based on its detection, in particular by a tracking system of the assistance system. The instrument further includes a second reference system arranged distally, in particular at the end section, in a predetermined, second condition relative to the working point, and which is designed as an optical, in particular planar, pattern adapted so that the position and/or orientation of the end section with the working point can be determined based on its optical detection, in particular by an optical recording system of the assistance system.

### CROSS-REFERENCE TO RELATED APPLICATION

This application claims priority under 35 U.S.C. § 119 to German Application No. 10 2023 112 754.3, filed on May 15, 2023, the content of which is incorporated by reference herein in its entirety.

### FIELD

The present disclosure relates to a medical, in particular surgical, instrument for a surgical assistance system(s) comprising: a proximal handling section, in particular a handle; a distal end section/end with a working point (located thereon), in particular a distal tip as distal working point, which is in particular adapted to palpate, aspirate, or navigate a tissue of a patient. The instrument has a first reference system proximally, which has a predetermined condition, that is, a predetermined position and orientation, relative to the distal end section with the working point, so that a position and/or orientation of the distal end section with the working point can be determined in three-dimensional space by means of detection of the first reference system. In addition, the present disclosure relates to a surgical assistance system, a method for determining the condition, i.e., the position and/or orientation, of the distal end section with the working point, and a computer-readable storage medium.

### BACKGROUND

In many surgical interventions, such as neurosurgery or spinal surgery, microscopic and endoscopic visualization or imaging and navigation of an instrument in the surgical intervention area are essential, operation-critical technologies. Both technological modalities, namely visualization on the one hand and navigation on the other, enable the surgeon to target, safely and precisely identify, and manipulate a tissue, as well as to precisely reference the tissue on preoperative recordings of the tissue. This is particularly important for minimally invasive surgical interventions where the surgeon's direct view is severely restricted.

Conventional navigation of the surgical instrument is based on the detection and tracking of infrared markers, for example infrared reflector spheres, which are attached to the instrument as rigid bodies. A tracking camera positioned at a comparatively large distance of around one to three meters captures and tracks these markers or tracks them spatially (as spatial tracking). During the surgical intervention, a line of sight problem can occur if an object gets into the line of sight that extends from the tracking camera to the markers and the view of the tracking camera on the markers is at least partially obscured, making it impossible to navigate the instrument. Such an object is, for example, the surgical visualization system, i.e., the surgical endoscope or surgical microscope, or even a medical professional who moves into the line of sight. Since the tracking-based navigation of the instrument must be made possible again, the line of sight problem means that the visualization system has to be adjusted contrary to the needs of the surgeon and is also time-consuming. This adjustment clears the field of view of the tracking system and makes it possible to navigate the instrument again.

### SUMMARY

It is therefore the tasks and objectives of the present disclosure to avoid or at least minimize the disadvantages of the prior art and to provide a medical instrument, a surgical assistance system, a (condition determination) method and a storage medium, each of which enables reliable and safe, in particular interference-free, tracking of the instrument. A partial task with regard to the surgical assistance system can be seen in ensuring reliable and trouble-free tracking and navigation of the instrument with as little technical effort as possible and solving or at least reducing the above-mentioned line of sight problem so that additional manual effort and disruptions during a medical/surgical intervention no longer occur or are at least reduced.

In principle, therefore, a medical instrument is provided that has at least two differently arranged, detectable reference systems. In this way, the instrument is adapted so that its tracking in three-dimensional space—in other words, the determination of its condition or position and orientation—can be carried out on the basis of (at least) two redundant reference systems, which in particular provide two different detection modalities. On the one hand, redundancy can be used to determine the condition of the instrument even if one of the reference systems is no longer sufficiently detectable, for example if it is covered, shielded or defective. On the other hand, the redundancy of the reference systems can be used to increase the accuracy of the condition determination if more than just one reference system is sufficiently detectable.

In a specific embodiment, the medical, in particular surgical, instrument is intended for a surgical assistance system. It has a proximal handling section, in particular a handle for manual guidance or a handling section for connection to a robot, and a distal end section with a distal working point, in particular a distal tip as the distal working point. The position of the working point is—in addition to the orientation of the instrument in space—the relevant variable for navigating the instrument. The position of the working point and the orientation of the instrument thus clearly define the condition of the instrument. Preferably, the instrument has a connecting section, in particular a shaft, through which the proximal handling section is connected to the distal end section. In particular, the instrument is adapted to be inserted into a patient intracorporeally and/or placed on the patient, for example in the form of a rigid endoscope, the distal section of which can be inserted into a patient intracorporeally. For tracking the condition of the instrument, in particular a position of the working point and an orientation (of a distal section) of the instrument, the instrument has a detectable first reference system which is arranged proximally in a predetermined first condition relative to the distal working point. In particular, the first reference system is rigidly attached to the proximal handling section and adapted so that the position and/or orientation of the distal end section with the working point can be determined on the basis of its detection, in particular by a tracking system of the assistance system. According to the invention, the instrument also has a second reference system which is arranged distally, in particular at the distal end section, in a predetermined second condition relative to the distal working point. The second reference system is designed as an optical, in particular planar, pattern (in particular a two-dimensional pattern) and is adapted so that the position and/or orientation of the distal end section with the working point can be determined on the basis of its optical detection, in particular by an optical recording system of the assistance system.

In other words, a medical instrument is provided which has at least two differently arranged (located), detectable reference systems, which preferably also differ with respect to a detection modality. In particular, the different location of the reference systems means that one reference system is still available for tracking if the other reference system is shielded, obscured or defective, and vice versa. This enables the condition of the instrument to be determined reliably and without interference. In addition, the at least two reference systems make it possible to increase the accuracy with which the condition can be determined. Furthermore, a first tracking mode or modality can be used in particular by a first detection modality, whereas a second detection modality can be used in particular by a second tracking mode or modality, which is more suitable in certain situations than the first modality.

The number of reference systems to be recorded by the instrument is of course not limited to two. The instrument has exactly two reference systems or at least two reference systems.

Advantageous embodiments are explained in particular below.

Preferably, the first and second reference systems are hybrid. The term hybrid means that different recording technologies or recording modalities are assigned to the reference systems. In other words, the first reference system can have a first detection modality and the second reference system can have another, different detection modality.

According to a further development, the first reference system can be designed as a rigid body, in particular with at least three, preferably four, reference bodies distributed in a plane. The reference bodies can be designed in the form of infrared-reflecting spheres (IR spheres) or in the form of LEDs, in particular in order to be detected by a tracking camera of the tracking system of the assistance system, in particular by an infrared tracking camera.

According to a preferred further development, the second reference system, in particular the optical pattern, extends along a surface or a surface section, in particular the distal end section. This means that no additional installation space is required for the second reference system and the instrument is not larger in the region of the second reference system than specified by its medical/surgical function. The handling of the instrument is therefore not affected by the second reference system, in particular not adversely. Since the second reference system is located at the distal end section that has the working point, it is preferably adapted to be captured by an optical recording system of the assistance system, in particular by a surgical microscope or a surgical endoscope, via which the surgical intervention can be tracked as intended anyway.

According to one variant, the optical pattern is regular in the direction of a working axis or longitudinal axis of the instrument. In a preferred, alternative embodiment, the optical pattern has a variation which, even if the pattern is partially obscured in this direction, makes it easier to determine the position and/or orientation of the distal end section with the working point.

It is particularly preferable for the pattern to consist of several individually and three-dimensionally differentiated elements or surface elements (plate-shaped) that extend in three-dimensional space. This means that just one of these elements can be detected by the recording system in order to clearly identify and analyze it using machine vision. The position and/or orientation of the end section with the working point can then be clearly determined from the identification and analysis by the data processing unit.

According to one variant, the optical pattern is regular in the peripheral direction of the working axis or longitudinal axis. In an alternative variant, it has a variation in the peripheral direction, by the detection of which a rotation of the distal end section with the working point about the working axis or longitudinal axis can be determined even if the pattern is partially covered in the peripheral direction and in particular if the instrument is rotationally symmetrical in the region of the pattern.

The optical pattern can, for example, have a barcode or a bar grid. Alternatively or additionally, it is formed by geometric shapes or geometries, in particular regular geometries, which are arranged in the form of a matrix or table. Standard geometries include polygons, circles, ovals, and the like.

Basically, it is advantageous in terms of detectability if the pattern is designed with the greatest possible contrast between the lines, grid, geometries, or regular geometries. A two-tone pattern proves to be simple and advantageous. The optical pattern formed in particular from complementary colors is preferred due to the resulting strongest contrast. In particular, white and black or a light color or color perception and a dark color or color perception should be mentioned here. For example, a light color can be created by keeping the surface structure “smooth”, while an engraving or corrugation or roughening of a surface, on the other hand, causes partial absorption of light and is therefore perceived as dark. This allows an optical pattern to be created sustainably by processing a surface without applying a color. One of the colors, and thus a region of the optical pattern, can be formed, for example, by the surface of the instrument that is not treated separately in terms of color. The other color, and thus the other region of the optical pattern, can be designed in complementary colors, for example by means of printing, laser printing, etching or another surface treatment.

Preferably, the medical instrument is designed as a navigation pointer or as an endoscope or it is designed in the form of a suction tube.

The term “distal” in the present disclosure defines a side or direction (towards the patient) facing away from the user, such as a surgeon. In contrast, the term “proximal” defines a side or direction facing towards the surgeon (away from the patient).

The term “position” refers to a geometric position in three-dimensional space, which is specified in particular by means of coordinates of a Cartesian coordinate system. In particular, the position can be specified by the three coordinates X, Y, and Z.

The term “orientation” in turn indicates an orientation (such as position) in space. It can also be said that the orientation indicates a direction or rotation in three-dimensional space. In particular, the orientation can be specified using three angles.

The term “condition” comprises both a position and an orientation. In particular, the condition can be specified using six coordinates, three position coordinates X, Y and Z and three angular coordinates for the orientation.

The term “distal end section” means a distal region/section of the instrument which is in particular 25%, particularly preferably 10%, most preferably 5%, of the total length of the instrument (from the proximal end of the handling section to the distal tip) along the longitudinal axis from the handling section to the distal tip. The distal end section can either be a separate assembly of the instrument or a section of the shaft itself.

According to a further aspect of the disclosure, a distance (“total length”) between the proximal end of the handling section and the working point may be less than 50 cm, particularly preferably less than 40 cm and most preferably less than 30 cm.

In particular, the second reference system can be a matrix-shaped optical pattern with at least four pattern elements, of which at least two pattern elements are designed differently, extending completely around the distal end section of the instrument and in the longitudinal axis direction. In other words, “plate elements” are used as pattern elements, so to speak, which are mounted on or inserted into a cylindrical surface.

The problems of the present disclosure are solved with respect to a surgical assistance system for use in a surgical intervention on a patient in that the surgical assistance system comprises an optical recording system, in particular with a surgical microscope and/or a surgical endoscope, which is preferably connected to a robot arm and which is adapted to create and provide at least one up-to-date recording of a surgical intervention area of a patient; a medical, in particular surgical, instrument according to the disclosure, which, as intended, is arranged at least with its distal end section with the working point in a field of view of the recording system defining the up-to-date recording or can at least be arranged there; a tracking system adapted to detect at least the first reference system of the instrument; and a data processing unit adapted to determine a position and/or orientation of the distal end section with the working point at least on the basis of the detected first reference system. According to the disclosure, the data processing unit is further adapted to determine the position and/or orientation of the distal end section with the working point on the basis of the second reference system depicted in the up-to-date recording, in particular by means of a machine vision or machine vision algorithm, which is preferably stored for execution in the data processing unit.

In this way, in addition to the modality formed by the first reference system and the tracking system, an additional modality is provided via which the position and/or orientation of the distal end section with the working point can be determined, so that redundancy is provided in the determination. This redundancy is provided with little technical effort by locating the two reference systems differently, once proximal and once distal to the instrument. In this way, navigation of the instrument is ensured even if one of the reference systems is obscured, shielded, or damaged, so that the above-mentioned “line of sight problem” can occur with significantly less frequency and thus the additional manual effort and disruption of the surgical intervention based on the “line of sight problem” are reduced accordingly.

According to a further development, the first reference system is designed as a rigid body with preferably at least three, in particular four, distributed reference bodies. The reference bodies are arranged in a plane and are in particular spherical infrared reflector bodies (IR bodies).

In this embodiment, the tracking system for tracking the reference bodies preferably has a 3D camera system with at least one 3D camera or a stereo camera and is adapted to detect/track the infrared reflector bodies (IR bodies). The data processing unit is adapted to determine the position and/or orientation of the distal end section with the working point relative to the tracking system by means of the detected first reference system and its predetermined condition relative to the distal end section with the working point.

In particular, the predetermined condition of the first reference system relative to the distal end section with the working point is stored in the data processing unit in the form of a first transformation matrix and the data processing unit can access this transformation matrix for determination, so that the data processing unit can determine the position and/or orientation of the distal end section with the working point relative to the tracking system by means of a simple mathematical calculation from the detection of the first reference system.

In particular, the tracking system has a rigid body with infrared reflector bodies that can be detected by a 3D camera system of the tracking system, so that the condition of the first reference system relative to the 3D camera system of the tracking system can be determined. In particular, exactly four IR spheres are attached to the instrument as rigid bodies.

According to an alternative embodiment, the first reference system may comprise electromagnetic sensors (EM sensors) and the tracking system may comprise an electromagnetic sensor system. In one variant, the electromagnetic sensors of the first reference system are adapted to detect an electromagnetic field generated by the tracking system and to determine a condition of the first reference system relative to the tracking system on the basis of this detected field. Alternatively, the electromagnetic sensors of the first reference system can generate two or three perpendicular electromagnetic fields which can be detected by a tracking system designed as an electromagnetic sensor system in order to determine the condition of the first reference system in three-dimensional space relative to the tracking system on the basis of the detected fields. In particular, the geometric first reference system can therefore have electromagnetic sensors that are tracked via an electromagnetic sensor system.

Preferably, the assistance system has a robot base as a local connection point of a robot comprising the above-mentioned robot arm. The robot arm is preferably movable and preferably has at least one articulated robot arm segment.

In order to make the determination of the position and/or orientation of the distal end section with the working point as robust as possible with respect to masking, shielding or defects in the reference systems, the data processing unit is adapted according to a further development to determine the position and/or orientation of the distal end section with the working point on the basis of the detection of either the first reference system or the second reference system or on the basis of the detection of both reference systems.

The first two cases mentioned above ensure the determination even if one of the reference systems and/or the associated system for its detection—i.e., the tracking system or the recording system-fails due to masking, shielding or a defect.

The latter case, in which both reference systems can be detected, can be used to improve the accuracy of determining the position and/or orientation of the distal end section with the working point.

For this purpose, an optimization algorithm is preferably stored in the data processing unit for execution, by means of which the determination accuracy can be increased. The optimization algorithm can, for example, be such that a position and/or orientation is determined on the basis of the respective reference system. Based on this, an average value of the positions and/or orientations can be determined, which is then finally incorporated into the navigation of the instrument as the valid, determined position and/or orientation of the distal end section with the working point. In this way, the data processing unit is adapted to determine the position and/or orientation of the distal end section with the working point with minimum error.

According to a possible further development of the assistance system, the tracking system is adapted to also detect the second reference system of the instrument and, alternatively or additionally, the recording system is adapted to also detect the first reference system of the instrument. In this way, at least the security against failure of the respective detection system is further increased.

According to a further development of the assistance system, the data processing unit is adapted to determine a condition of the recording system on the basis of stored kinematics of the robot and at least one recorded joint angle of the robot arm. In other words, the assistance system has a robot kinematics-based tracking device that can detect the condition of the recording system, in particular the camera, based on the current configuration of the robot arm. In this way, the condition of the instrument relative to the robot base and also relative to the patient can ultimately be determined, in particular by serially connecting a tracking of the robot head with the recording system on the one hand (relative to the robot base, wherein a transformation between the robot base and the patient is known in particular by means of a registration) and the tracking of a condition of the instrument relative to the robot head (with the recording system).

Alternatively or additionally, a rigid body detectable by the recording system or electromagnetic sensors or electromagnetic transducers detectable by the recording system are arranged on the outside of the mounting system, wherein the data processing unit is adapted to determine a condition of the recording system on the basis of this detected rigid body or these detected electromagnetic sensors or transducers.

With regard to a method for determining a condition of the instrument, in particular in a surgical assistance system according to the present disclosure, the tasks and objectives of the present disclosure are solved in that the method comprises:
An arrangement of the instrument, preferably at least in sections in a field of view of a recording system, in particular a surgical microscope and/or a surgical endoscope of the assistance system, and preferably in a surgical intervention area of a patient. It is of course preferable that the recording system is arranged in such a way that its field of view includes the surgical intervention area, at least in sections;
The recording system creates an up-to-date recording. The recording preferably contains at least in sections, the surgical intervention area and the instrument located there. Preferably, this recording is displayed on a display unit of the assistance system, such as a monitor or VR glasses, so that the surgeon or other medical personnel can follow the surgical intervention;
a recording system provides the up-to-date recording, in particular in digital form;
a tracking system of the assistance system detects the first reference system of the instrument, in particular in real time; and
an, in particular up-to-date, determination of a position and/or orientation of the distal end section with the working point, preferably a position of the working point and an orientation of the distal end section, on the basis of the detected first reference system by a data processing unit of the assistance system; according to the invention, the method further comprises:
an optical detection of the second reference system of the instrument, in particular in real time, by the up-to-date recording of the recording system; and
an, in particular, up-to-date, determination of the position and/or orientation of the distal end section with the working point, in particular at least the position of the working point, on the basis of the detected second reference system by machine vision or machine vision by the data processing unit.

Thus, according to the method, at least two different reference systems are detected on the one instrument by one detection system each, once by the tracking system and once by the recording system. The determination of the position and/or orientation of the distal end section with the working point, preferably the position of the working point and the orientation of the distal end section, thus exhibits redundancy and thus proves to be particularly robust against the line of sight problem described above.

According to a further development, the method has continuous steps:
determining whether the first reference system is adequately detected by the tracking system; determining whether the second reference system is adequately detected by the recording system; and depending on a determination result, determining the position and/or orientation of the distal end section with the working point on the basis of only the first reference system or on the basis of only the second reference system or on the basis of both reference systems. The three steps mentioned are preferably carried out by the data processing unit.

A criterion for sufficient detectability with regard to the first reference system is preferably its complete detectability. In particular, this means that the data processing unit determines whether all markers, bodies, and sensors of the first reference system are detected by the tracking system. If this is the case, the first reference system is considered to be sufficiently detectable and is used to determine the position and/or orientation.

With regard to the second reference system, the criterion depends on the structure of the optical pattern. If the optical pattern is made up of several individually and three-dimensionally differentiated elements, as shown above, the complete detection of just one of these elements is sufficient to be able to clearly identify and analyze it using machine vision. The position and/or orientation of the end section with the working point, in particular the position of the working point and the orientation of the distal end section, can then be clearly determined from the identification and analysis.

With respect to a computer-readable storage medium, the problems and objectives of the present disclosure are solved in that the computer-readable storage medium comprises instructions which, when executed by a computer, cause the computer to perform the method steps of the method for determining the position and/or orientation according to the present disclosure.

The features of the medical instrument of the present disclosure and the surgical assistance system of the present disclosure may be interchanged.

### BRIEF DESCRIPTION OF THE DRAWING FIGURES

The present disclosure is explained below with reference to preferred embodiments with the aid of accompanying figures.

FIG. 1 shows a perspective view of a medical instrument in the form of an endoscopic navigation pointer according to a first embodiment, in which a first reference system is arranged at the handling section and a second reference system is arranged at the distal end section;

FIG. 2 shows a perspective view of a medical instrument in the form of a navigation pointer according to a second embodiment, in which a first reference system is arranged at the handling section and a second reference system is arranged at the distal end section;

FIG. 3 shows a perspective detail view of a distal end section of an endoscopic navigation pointer according to a third embodiment with a second reference system;

FIG. 4 shows a schematic view of a medical assistance system with the instrument according to FIG. 1 according to a preferred embodiment; and

FIG. 5 shows a flow diagram of a method for determining a condition of the instrument according to a preferred embodiment.

The figures are merely schematic in nature and are only intended to aid understanding of the publication. The features of the various embodiments can be interchanged.

### DETAILED DESCRIPTION

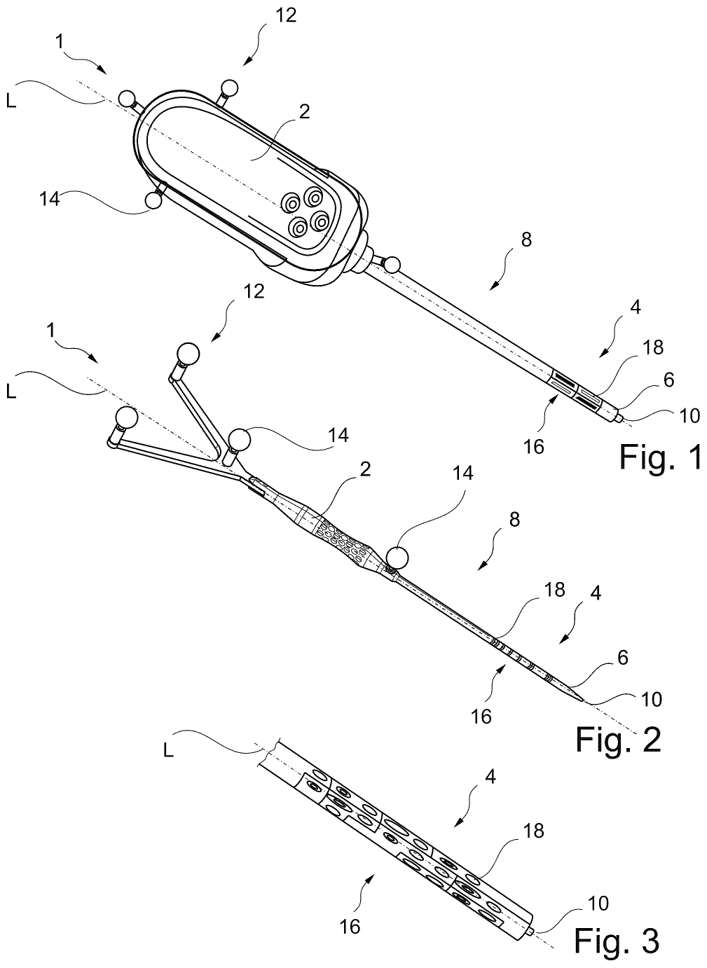

FIG. 1 shows a perspective view of a medical instrument according to a preferred embodiment, which is designed as a palpation-capable, endoscopic navigation pointer/endoscopic navigation pointer/endoscope navigation pointer 1. It can also be said that this medical instrument is designed as a rigid endoscope, which can also perform a palpation function in particular if required and thus, in addition to the endoscope recording with corresponding condition determination of the recording, also enables punctual position determination with the endoscope tip of the rigid endoscope shaft, for example to mark landmarks.

The endoscopic navigation pointer 1 according to FIG. 1 has a proximal handling section in the form of a handle 2, which is adapted to be gripped and guided manually by a member of medical personnel such as a surgeon. Alternatively, the handling section can also be adapted to be guided by a robot arm.

A distal end section 4 of the endoscopic navigation pointer 1 is adapted to be inserted into or placed on a patient intracorporeally and has a blunt distal tip 6 adapted to create an intracorporeal recording, for example to provide the surgeon with a up-to-date tissue recording, wherein this tissue recording can be localized in a navigation system and furthermore, preferably, to precisely and selectively scan or palpate an intracorporeal tissue of the patient as required without irreversibly damaging the tissue. tissue without irreversibly damaging the tissue.

A rigid shaft 8 rigidly connects the distal end section 4 and the handle 2 and has an essentially cylindrical and closed outer contour along a longitudinal axis L. In the embodiment shown, the distal end section 4 is a distal subsection of the shaft 8 and has the working point 10, which is relevant for navigation of the instrument 1 and is arranged on the longitudinal axis L, on the end face of its distal tip 6.

The handle 2 has a first reference system 12 with four rigidly connected infrared reflector markers 14 (IR spheres) arranged together in one plane, of which only one is marked with a reference symbol in FIG. 1 for reasons of clarity. The distal end section 4 with the distal tip 6 and the working point 10 have a rigid geometric (first) relative condition in relation to the first reference system 12. By determining the condition of the first reference system 12, i.e., determining its position and orientation, the condition of the distal end section 4 and the distal tip 6 and thus also the position of the working point 10 can be easily determined. It can also be said that a transformation matrix between the working point 10 and the first reference system 12 is predetermined and static, so that a corresponding determination of the condition of the distal end section 4 in three-dimensional space can be determined via the detection of the first reference system 12.

This design with distal tip 6, shaft 8 and handle 2 with first reference system 12 essentially corresponds to the design of a navigation pointer/navigation pointer, which is used to aim at a specific point in three-dimensional space by means of the working point 10 and to detect it geometrically in three-dimensional space in relation to 3D recording data.

According to FIG. 1, the instrument 1 has a further, second reference system 16 at its distal end section 4, which is designed as an optically detectable surface pattern. In the exemplary embodiment shown, the optical pattern extends fully along the distal end section 4 with rows of geometric elements 18 spaced along the longitudinal axis L. The geometric elements 18 extend parallel to the longitudinal axis L as elongated rectangles, which alternately have a black and a white coloring (or a dark and light coloring) in the peripheral direction. Due to this variation of the pattern, both in the longitudinal and peripheral direction, the position of the working point 10 and the complete orientation of the instrument 1, i.e., also its rotation around the longitudinal axis L, can be determined by the detection of the second reference system 16 according to FIG. 1.

The second reference system 16 has a rigid geometric (second) relative condition to the distal end section 4, the distal tip 6 and the working point 10. It is therefore also true for the second reference system 16 that a corresponding determination of its condition enables the condition of the distal end section 4 to be determined.

In this way, the spatial condition of the endoscope tip (instrument tip) can be recorded as a working point by means of a tracking camera and the IR markers IR spheres in order to determine the actual position and/or orientation of the recording in particular, as well as to use the position of the instrument tip itself for landmark detection, for example, or the optical pattern and thus also the position and preferably also the orientation can be determined very precisely by means of a surgical microscope (which is arranged very close to the instrument tip, for example only 15 cm away). The first and second reference systems can be used redundantly as well as optionally and improve condition detection.

FIG. 2 shows a second exemplary embodiment of a medical or surgical instrument 1 according to the invention. The instrument 1 shown here is designed as a navigation pointer, wherein the description of FIG. 2 will only focus on the differences to the exemplary embodiment shown in FIG. 1.

In contrast to the navigation pointer shown in FIG. 1, the distal tip 6 shown in FIG. 2 is actually designed as a very fine tip and is therefore particularly suitable for very precise navigation and referencing of tissue with reference to, for example, preoperative 3D recordings of the patient (pointer).

The first reference system 12 according to FIG. 2 also consists of infrared reflector markers 14, which, however, have a slightly different arrangement than in FIG. 2. If the markers 14 were arranged in the plane in the form of a cross according to FIG. 1, they are arranged at the end points and at the intersection point of a Y according to FIG. 2, wherein the handle 2 extends approximately from the marker 14 arranged at the intersection point to the marker 14 arranged at the base point of the Y.

The second reference system 16 according to FIG. 2 is also designed as an optically detectable surface pattern that extends fully around the longitudinal axis L at the distal end section 4. The geometric elements 18 of the pattern are simple circular rings with the same line width. The circular rings 18 are combined into groups along the longitudinal axis L, within which the circular rings are comparatively close together, whereas the distance between the groups increases towards the working point 10. Due to the fact that the pattern formed by the circular rings 18 shows no variation in the peripheral direction, the position of the working point 10 and the orientation of the instrument 1 can be determined by the detection of the second reference system 16 according to FIG. 2, but not its rotation around the longitudinal axis L.

With the navigation pointer in FIG. 2, the position of the instrument tip (as working point) can also be determined both via the first reference system and via the second reference system, with the same advantages as described above.

FIG. 3 shows a detailed perspective view of a third exemplary embodiment of an instrument with a second reference system 16 at the distal end section 4 with a predetermined condition relative to the working point 10, wherein the distal end section 4—similar to that shown in FIG. 1—is comparatively blunt and is suitable for palpation. In particular, this design can be provided at the distal end section shown in FIG. 1. The elements 18 of the optical pattern of the second reference system 16 are formed by single or multiple nested ovals of different colors. The ovals 18 are distributed around the entire periphery of the distal end section 4 in rows spaced along the longitudinal axis L. The ovals 18 extend longitudinally in the direction of the longitudinal axis L and are designed in the peripheral direction alternately on a dark and on a light background in the respective contrasting color.

In the exemplary embodiment shown in FIG. 3, the position of the working point 10 and the complete orientation of the instrument 1, i.e., also its rotation about the longitudinal axis L, can also be determined by detecting the second reference system 16 due to the variations of the pattern 16 in both the longitudinal and peripheral directions.

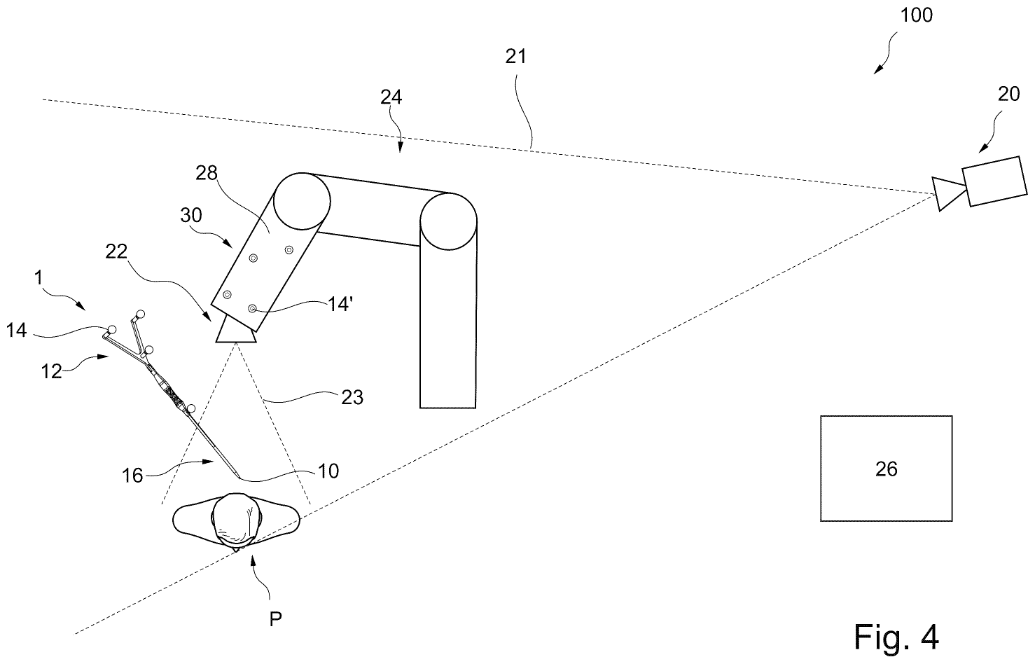

FIG. 4 shows a medical or surgical assistance system 100 in schematic form. The assistance system 100 has a tracking system with an infrared emitter (not shown) and a 3D camera 20. The camera 20 has a first field of view 21 for detection, in which the patient P, the instrument 1 and a robot arm 24 with an end segment 28, which carries a recording system 22, are arranged.

The infrared emitter irradiates the infrared reflector markers 14 of the first reference system 12 and the 3D camera 20 captures the four infrared reflector markers 14. On the basis of this acquisition, a data processing unit 26 of the assistance system 100 determines the condition of the first reference system 12 relative to the 3D camera 20 of the tracking system and, using the transformation of the first reference system 12 relative to the working point 10 stored therein, the data processing unit 26 determines the position of the working point 10 and the orientation of the instrument 1 relative to the 3D camera 20 and thus to the tracking system.

The optical recording system 22, which in the present exemplary embodiment has a surgical microscope, has a second field of view 23 in which the patient P, the working point 10 and at least the distal end section of the instrument 1 with the second reference system 16, which is designed as an optical pattern, are arranged.

The recording system 22 continuously creates an up-to-date recording of the surgical intervention area of patient P defined by the second field of view 23 and also captures the second reference system 16 with its optical pattern with each recording (see 18, FIGS. 1 through 3).

The data processing unit 26 has a machine vision algorithm stored for execution, with which it analyzes the captured optical pattern of the second reference system 16 and determines the condition of the second reference system 16 relative to the recording system 22. The data processing unit 26 uses the transformation of the second reference system 16 relative to the working point 10 stored in it to determine the position of the working point 10 and the orientation of the instrument 1 relative to the recording system 22.

On the end segment 28, to which the recording system 22 is rigidly connected, a rigid body 30 is placed on a housing surface, which is formed by four infrared reflector markers 14′ irradiated by the infrared emitter and detected by the 3D camera 20 of the tracking system. Based on their detection, the data processing unit 26 determines the condition of the rigid body 30 relative to the 3D camera 20 of the tracking system. By means of a transformation of the rigid body 30 relative to the recording system 22 stored in the data processing unit 26, the data processing unit 26 subsequently determines the condition of the recording system 22 relative to the 3D camera 20 and thus the condition of the recording system 22 relative to the tracking system.

The data processing unit 26 also stores digital, preoperative 3D recording data of patient P. These were recorded before the surgical intervention using magnetic resonance imaging (MRI) or computer tomography (CT), for example.

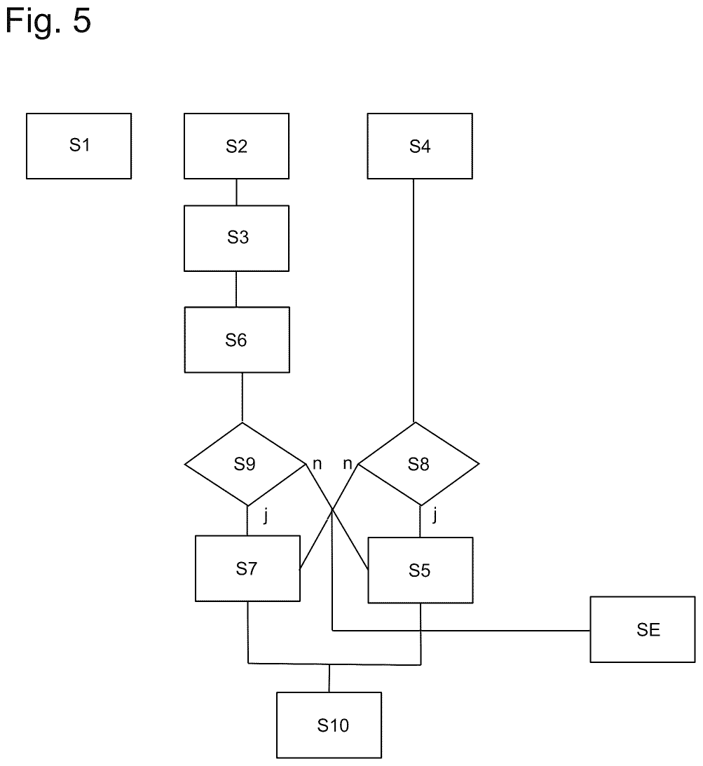

FIG. 5 shows a flowchart of a preferred exemplary embodiment of a method for determining the position and orientation of a medical instrument 1, in particular when it is used in the medical assistance system 100 according to FIG. 4. The method can, of course, be applied to all embodiments of the instruments 1 shown in FIGS. 1 through 3.

According to FIG. 5, an optional first step S1 takes place, in which the instrument is arranged in the field of view 23 of the recording system 22. A sensible prerequisite for this is, of course, that the recording system 22 with its field of view 23 has previously been aligned with the patient P's surgical intervention area. If this is not the case, the instrument 1 can of course be placed in the surgical intervention area and the field of view 23 of the recording system 22 is aligned with the surgical intervention area by corresponding control of the robot arm 24.

In step S2, the recording system 22 continuously creates an up-to-date recording of the surgical intervention area, in particular as a video stream. Due to its previous position in the field of view 23, the instrument 1 is shown at least in sections in the up-to-date recording.

In step S3, the recording is provided digitally and in real time by the recording system 22, for example to the data processing unit or on a display device of the assistance system 100.

Continuously, in step S4, the first reference system 12 of the instrument 1 is tracked by the tracking or 3D camera 20 capturing the infrared reflector markers 14, and in step S6, the second reference system 16 of the instrument 1 is tracked by the recording system 22 capturing the optical pattern of the second reference system 16.

In steps S8 and S9, the data processing unit 26 is used to determine whether the above-mentioned detections are made with sufficient quality, i.e., whether the respective reference system 12, 16 can be detected sufficiently well so that the respective determination of the position/orientation is possible from its detection with sufficient quality, i.e., whether a detection can be carried out at all and, if so, in particular within predefined error ranges or tolerance ranges.

The criterion for sufficient detectability of the first reference system 12 is preferably whether all four infrared reflector markers 14 can actually be detected by the 3D camera 20.

If this step S8 is positive (S8, j), the position of the working point 10 and the orientation of the instrument 1 are determined via the data processing unit 26 on the basis of the detected first reference system 12 in step S5.

The criterion for sufficient detectability of the second reference system 16 is preferably that at least one of the elements 18 of the optical pattern (see FIGS. 1 through 3) is clearly identifiable and detectable.

If this step S9 is positive (S9, j), the position of the working point 10 and the orientation of the instrument 1 are determined via the data processing unit 26 in step S7 on the basis of the detected second reference system 16 or on the basis of the detected section of the second reference system 16, i.e., in the limiting case of only one element 18.

If both reference systems 12, 16 are sufficiently detectable (S8, j and S9, j), the position and orientation are determined on the basis of the two detected reference systems 12, 16 in step S10. For example, errors are minimized by forming an average value from the two positions and orientations so that the position and orientation are optimally determined by the respective average value.

If one of the detections S4, S6 is not sufficient (S8, n or S9, n), the position and orientation can be determined on the other, sufficient detection.

In the event that none of the detections is sufficient (S8, n and S9, n), the data processing unit 26 outputs an error message in step SE.

The (condition determination) method allows the condition to be determined reliably and precisely.

### LIST OF REFERENCE SIGNS

1 Medical instrument
2 Handle
4 End section
6 Distal tip
8 Shaft
10 Working point
12 First reference system
14, 14′ Infrared reflector marker
16 Second reference system
18 Pattern element
20 Tracking system
21 First field of view
22 Visual recording system
23 Second field of view
24 Robot arm
26 Data processing unit
28 End segment
30 Rigid body
P Patient
L Longitudinal axis
S1 Step: Arrangement
S2 Step: Creation of the recording
S3 Step: Provision of the recording
S4 Step: Recording the first reference system
S5 Step: Determination
S6 Step: Recording of the second reference system
S7 Step: Determination
S7.1 Step: Determination based only on the first reference system
S7.2 Step: Determination based only on the second reference system
S7.3 Step: Determination based on both reference systems
S8 Step: Determination of sufficient detection of first reference system
S9 Step: Determination of sufficient detection of second reference system
S10 Determination of the position and/or orientation of the distal end section
SE Step: Error message

1. An instrument for a medical or surgical assistance system, the instrument comprising:
a proximal handling section;
a distal end section with a distal working point;
a first reference system arranged proximally in a predetermined first condition relative to the distal working point, the first reference system adapted for determining a position and/or an orientation of the distal end section with the distal working point; and
a second reference system arranged distally in a predetermined second condition relative to the distal working point, the second reference system designed as an optical pattern adapted to determine the position and/or orientation of the distal end section with the distal working point.

2. The instrument according to claim 1, wherein the first reference system is designed as a rigid body with reference bodies arranged in a distributed manner.

3. The instrument according to claim 1, wherein the second reference system extends along a surface or surface section of the distal end section to be detected by an optical recording system.

4. The instrument according to claim 1, wherein the optical pattern has a bar code and/or a bar grid and/or geometries or ruled geometries arranged according to a matrix.

5. The instrument according to claim 1, wherein the optical pattern is designed in two colors, in particular with maximum contrast.

6. The instrument according to claim 1, wherein the optical pattern is printed or laser-printed.

7. The instrument according to claim 1, wherein the instrument is designed as a navigation pointer or an endoscope, or wherein the instrument is designed as a suction tube.

8. An assistance system for a medical or surgical intervention, the assistance system comprising:
the instrument according to claim 1;
an optical recording system connected to a robot arm and adapted to create at least one up-to-date recording of a patient's surgical intervention area and to provide the at least one up-to-date recording in real time;
a tracking system adapted to detect at least the first reference system of the instrument; and
a data processing unit adapted to determine a position and/or orientation of the distal end section with the distal working point at least based on the first reference system,
wherein:
the data processing unit is further adapted to determine the position and/or orientation of the distal end section with the distal working point based on an up-to-date recording of the second reference system, and
the instrument being arrangeable as intended at least with the distal end section with the distal working point in a field of view of the optical recording system defining the at least one up-to-date recording.

9. The assistance system according to claim 8, wherein the data processing unit is adapted to determine the position and/or orientation of the distal end section with the distal working point based on the first reference system and/or the second reference system.

10. The assistance system according to claim 9, wherein the data processing unit is adapted to optimally determine the position and/or orientation of the distal end section with the distal working point based on the first reference system and the second reference system.

11. The assistance system according to claim 8, wherein the tracking system is further adapted to detect the second reference system of the instrument.

12. The assistance system according to claim 8, wherein the data processing unit is adapted to determine a condition of the optical recording system based on stored kinematics and at least one detected joint angle of the robot arm, and/or wherein a rigid body detectable by the tracking system is coupled to the optical recording system and the data processing unit is adapted to determine a condition of the optical recording system based on the rigid body.

13. A method for determining a position and/or orientation of an instrument of the assistance system according to claim 8, the method comprising the steps of:
arranging the instrument at least in sections in a field of view of a recording system;
creating an up-to-date recording by the recording system;
providing the up-to-date recording in real time by the recording system;
detecting the first reference system of the instrument by a tracking system;
determining a position and/or orientation of the distal end section with the distal working point at least based on the first reference system by a data processing unit;
optically detecting the second reference system of the instrument by the up-to-date recording; and
determining the position and/or orientation of the distal end section with the distal working point at least based on the second reference system by machine vision or machine vision of the data processing unit.

14. The method according to claim 13 further comprising the steps of:
determining whether the first reference system is adequately detected by the tracking system by the data processing unit;
determining whether the second reference system is adequately detected by the recording system by the data processing unit; and
depending on results of an investigation, determining:
a) the position and/or orientation of the distal end section with the distal working point based only on the first reference system; or
b) determining the position and/or orientation of the distal end section with the distal working point based only on the second reference system; or
c) determining the position and/or orientation of the distal end section with the distal working point based on both reference systems; or
d) outputting an error message that no determination can be made.

15. A computer-readable storage medium comprising instructions which, when executed by a computer, cause the computer to perform the steps of the method according to claim 13.
